(19) United States
(12) Patent Application Publication
(10) Pub. No.: US 2018/0333088 A1
(43) Pub. Date: Nov. 22, 2018

(54) Pulse Oximetry Capturing Technique

(71) Applicant: Microsoft Technology Licensing, LLC, Redmond, WA (US)

(72) Inventors: Christian Holz, Redmond, WA (US); Eyal Ofek, Redmond, WA (US)

(73) Assignee: Microsoft Technology Licensing, LLC

(21) Appl. No.: 15/597,514

(22) Filed: May 17, 2017

(51) Int. Cl. A61B 5/1455; H04N 7/18; G06T 7/90; G06T 5/20; A61B 5/00; A61B 5/103

(52) U.S. Cl. CPC A61B 5/14552; H04N 7/185; G06T 7/90; G06T 5/20; A61B 2576/00; A61B 5/742; A61B 5/1032; A61B 2562/0233; A61B 5/7278

(57) ABSTRACT

Embodiments relate to using a display and camera of a computing device to perform pulse oximetry. The display of the device is used as an illuminant, a finger is placed over a portion of the display and a camera facing in the same direction as the display. One or more colors are selected to enhance hemoglobin-deoxyhemoglobin contrast in view of display and camera sensitivities. The one or more colors are displayed while a body part covers the displayed color and the camera. The camera captures images of light that has passed through the finger and been internally reflected to the camera. The light reaching the camera has been absorbed by arterial hemoglobin and deoxyhemoglobin at different rates in respective different wavebands. Differences in attenuation of display light at the different wavebands provide sufficient contrast to compute an accurate pulse oxygenation estimate.

### BACKGROUND

Blood oxygenation is an important biomarker and accurate measurement of blood oxygenation is desirable for many health reasons. For example, regularly monitoring blood oxygenation can help detect cardiac and pulmonary conditions such as hypoxemia and sleep apnea. Athletes often use blood oxygenation measures to monitor their performance and improve their endurance training.

Pulse oximetry is one technique that has been used to measure blood oxygenation. Pulse oximeters leverage the differing light absorption rates of hemoglobin (oxygenated red blood cells) and deoxyhemoglobin (non-oxygenated red blood cells) at different wavelengths of light, typically, red and near-infrared. An oximeter typically includes a small measurement device clipped to a finger or ear lobe to measure peripheral arterial oxygen saturation. The device typically includes red and near-infrared light emission sources on one side of the finger, and light sensors on the other side. The light sensors measure the red and near-infrared light that has passed through the finger and uses the relative red and near-infrared light intensities to estimate oxygenation. While devices designed specifically for pulse oximetry are inexpensive and accurate (e.g., ±2-3%), they are single-purpose devices, because of the inconvenience of keeping a specialized device at hand, pulse oximeters are not often used by people without compelling reasons.

Unlike pulse oximeters, people often keep smartphones on their person or nearby. The potential to use smartphones as pulse oximeters without special hardware has been considered. The main solution to date has been to use a smartphone's photography flash as an illuminant in combination with the smartphone's rear-facing camera. A finger is placed over both the flash and the camera, white light from the flash passes through the finger and some is reflected to the camera. The camera signal is processed to estimate oxygenation. Although this technique provides an accurate measure of heartrate, oxygenation measures are unreliable for several reasons. Most smartphone cameras have integrated block filters which minimize optical sensitivity in the near-infrared region. Much of this filter-blocked region of light happens to include wavebands where deoxyhemoglobin reflects more light than oxyhemoglobin. Consequently, due to near elimination of sensing in these high-contrast bands, and due to the roughly uniform spectrum of flash light, flash light reflections from oxyhemoglobin and deoxyhemoglobin have low contrast and therefore result in less precise measures. Another approach has been to equip smartphones with additional hardware illuminants (e.g., light emitting diodes) and/or sensors, but low utilization of such hardware, the amount of cost it adds, and the additional hardware footprint might not be justified.

Techniques for using a computing device to measure pulse oximetry are discussed below.

### SUMMARY

The following summary is included only to introduce some concepts discussed in the Detailed Description below. This summary is not comprehensive and is not intended to delineate the scope of the claimed subject matter, which is set forth by the claims presented at the end.

A computing device has a display and a camera. The display emits light comprising a first waveband component and a second waveband component. The light from the display transmits through matter and is reflected to the camera. The reflected display light has a first waveband component and a second waveband component. Image data from the camera provides a first intensity corresponding to the first waveband component and a second intensity corresponding to the second waveband component. In one embodiment, a ratio of the first intensity and the second intensity are used to determine a property of the matter. Other embodiments may use other functions that involve the intensities of two or more bands of illumination. The technique may be used to measure relative ratios (or other functions) of any light-transmitting constituents of the matter. If the matter includes pulsing blood, the ratio corresponds to blood oxygenation.

Many of the attendant features will be explained below with reference to the following detailed description considered in connection with the accompanying drawings.

### BRIEF DESCRIPTION OF THE DRAWINGS

The present description will be better understood from the following detailed description read in light of the accompanying drawings, wherein like reference numerals are used to designate like parts in the accompanying description.

FIG. 1 shows a computing device.

FIG. 2 shows a process for computing a ratio of constituent components through which light from a display has passed before being received by a camera.

FIG. 3 shows absorption curves, display brightness curves, and camera sensitivity curves.

FIG. 4 shows examples of raw and filtered color signals.

FIG. 5 shows details of a computing device on which embodiments described herein may be implemented.

### DETAILED DESCRIPTION

Embodiments discussed below relate to using a display and camera of a computing device to measure pulse oximetry. The display of the device is used as an illuminant, in one embodiment a finger is placed over a portion of the display and a camera facing in the same direction as the display (e.g., a front-facing camera of a smartphone). One or more colors are selected to enhance hemoglobin-deoxyhemoglobin contrast in view of display and camera sensitivities. The one or more colors are displayed while a finger or other body part covers the displayed color and the camera. The camera captures images of light that has passed through the finger and been partially internally reflected to the camera. The light reaching the camera has been diminished by absorption by arterial hemoglobin and deoxyhemoglobin at different rates in respective different wavebands. Differences in attenuation of display light at the different wavebands provide sufficient contrast (ratio R) to compute an accurate pulse oxygenation estimate (e.g., commonly by using a lookup table).

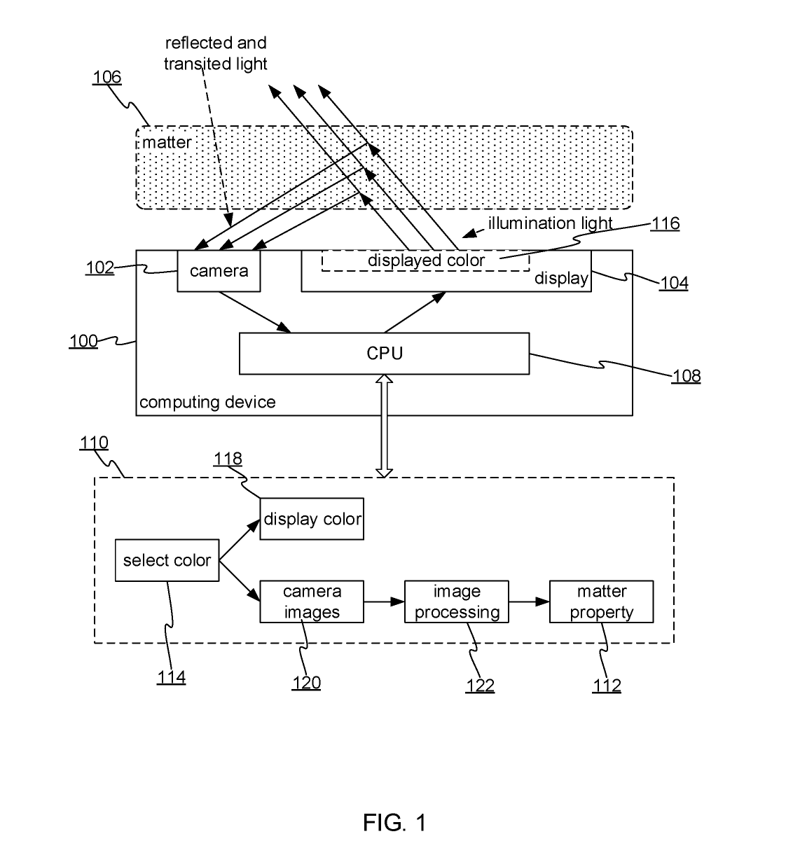

FIG. 1 shows a computing device 100. The computing device 100 includes a camera 102 and display 104. Preferable, the camera 102 and display 104 are arranged to allow a body of matter 106 to be near (or cover) both the display 104 and the camera 102 such that light emitted by the display 104 will transmit through the matter 106 and at least in part be reflected to the camera 102. The matter 106 includes at least two constituent components which have different light absorption properties at different wavebands. The matter 106 may contact the computing device, may be near the computing device, or the computing device may be immersed in the matter (in the case of a liquid or gas). The camera 102 and display 104 may be ordinary stock or mass-produced consumer grade items; hardware with unusual optical properties is not necessary.

The computing device 100 also includes processing hardware 108 to execute a process 110 for determining a property 112 of the matter 106. The process 110 may be an application executed by the computing device's operating system or other such software. Initially, an illuminant color is selected 114. Consider that there are high-contrast wavebands where the constituent components of the matter 106 have different respective absorption rates (typically in bands between isosbestic points). The illuminant color is selected to maximize illumination at these high-contrast bands. The illuminant color 116 is displayed 118 by the display 104. The light from the displayed color 116 transits through the matter 106 and is partially reflected to the camera 102. While the color 116 is displayed, the camera captures one or more images 120 of the light from the display 104 that has both transited through the matter 106 and reflected to the camera. The images 120 include two or more color channels. As described later, the images 120 are processed 122 to extract whichever color channels are appropriate for contrast-sensitive wavebands of the elements in the optical pathway (i.e., the display, matter, and camera).

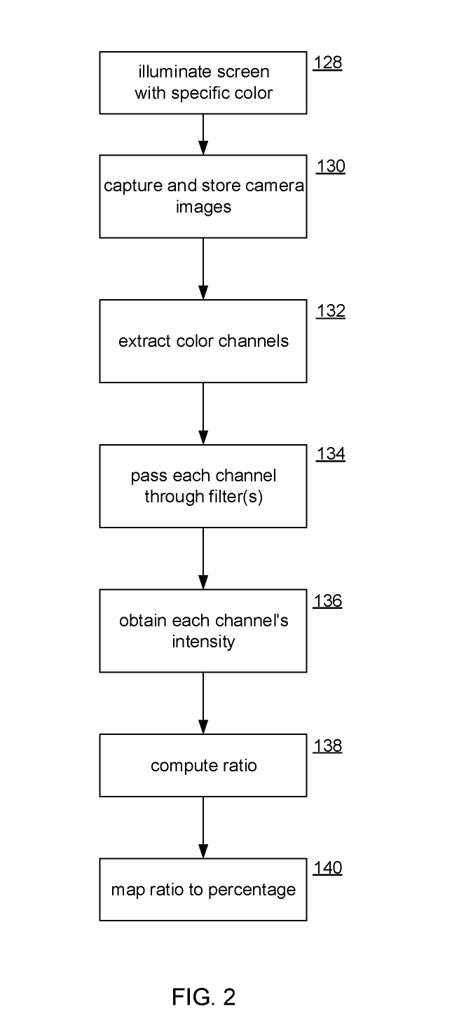

FIG. 2 shows a process for computing a ratio of constituent components through which light from the display 104 has passed before being received by the sensor of the camera 102. While pulse oximetry is a practical application of the process, any body of matter with constituent elements, compounds, etc. having sufficiently varying absorption profiles can be measured with the process of FIG. 2. As noted above, while a color is displayed (step 128) and a body is placed in proximity to the camera and display, at step 130 the camera captures a sequence of still images or encoded video. Preferably, the color is chosen to maximize overlap of targeted high-contrast bands of the first and second constituents and bands resolved by the camera. The targeted high-contrast bands may include (i) a first band where the first constituent generally has higher absorption than the second constituent, and to include (ii) a second band where the second constituent generally has higher absorption than the first constituent.

At step 132 target color channels are extracted from the captured video/image sequence. The result is a raw time-domain intensity signal for each color. At step 134 each target color signal is passed through one or more filters for noise reduction, etc. At step 136 a statistical measure of intensity is obtained for each target color signal. The statistical measure may be any type of statistical aggregation such as arithmetic mean, harmonic mean, geometric mean, root means squared, average, etc. Different statistical measures might be taken for the respective target color signals, for different time periods, for different signal components, etc. For discussion, it will be assumed that the first color signal yields a statistical intensity for each respective target color signal. At step 138 a ratio of the intensities is computed, and at step 140 the ratio is applied to a table or function that maps the ratio to relative proportions of the constituent components. It is also possible to use other functions of the intensities to identify the composition of the measured matter. Any function that meaningfully varies with varying intensities of the color signals may be considered.

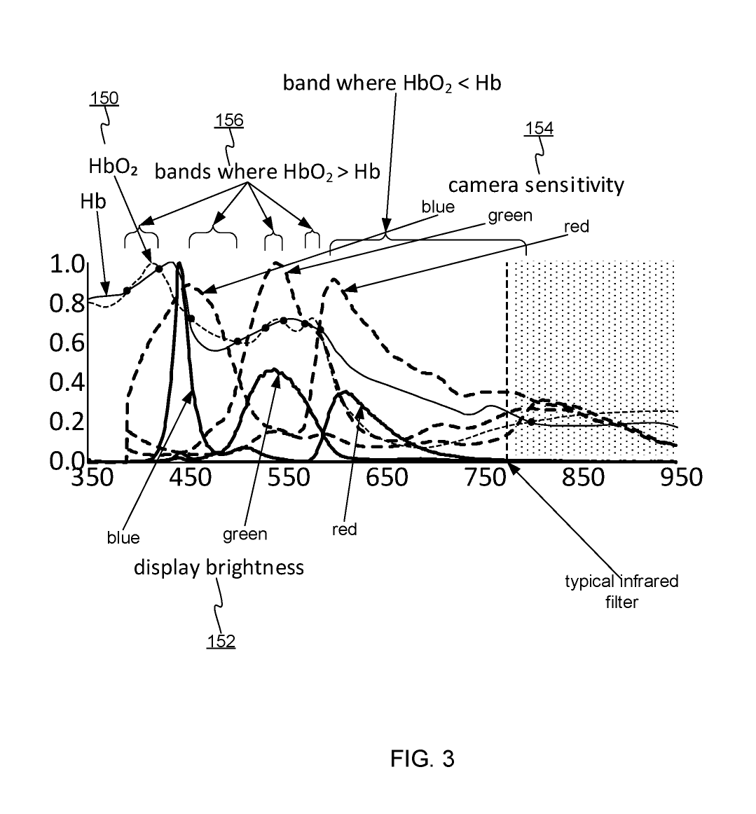

An embodiment for implementing pulse oximetry on a smartphone with stock hardware is not described. FIG. 3 shows absorption curves 150, display brightness curves 152, and camera sensitivity curves 154. The absorption curves 150 are for oxyhemoglobin (HbO₂, thin dashed line) and deoxyhemoglobin (Hb, thin solid line). The isosbestic points are the dots where the absorption curves intersect. By computing intensities at or within one or more isosbestic bands 156 and at or within one or more non-isosbestic bands, the corresponding camera color intensities provide sufficient signal contrast for a blood oxygenation estimate. Note that the isosbestic bands 156 include the bands where oxyhemoglobin has higher absorption than deoxyhemoglobin and bands where the reverse is true.

While embodiments are described for emitting two color channels, depending on the material being measured and the profiles of the camera and display, accuracy might be higher if three color channels are displayed (either uniformly or non-uniformly, as circumstances suggest). Similarly, more than two color channels of the images may be used for higher accuracy. Furthermore, although this description mentions selecting one or more colors for illumination, an automated decision-making process to identify ideal colors is not required. For applications intended for a known material (e.g., blood and tissue), the particular colors to be displayed and/or analyzed for intensity may be hard-coded to be specific to the material. In another embodiment, there may be an incremental walk through the camera/display spectrum with sampling and analysis performed across many wavebands of the spectrum, which can reveal wavebands where there is maximal contrast. In yet another embodiment, a user interface may allow a user to specify the target material and target colors are set accordingly during runtime.

As can be seen in FIG. 3, red and green are the colors where camera sensitivity, display output, and isosbestic bands best overlap. Specifically, FIG. 3 shows that the green illumination peak overlaps with an area in which deoxyhemoglobin reflects more light than oxyhemoglobin. FIG. 3 also shows that in the red spectrum, the illuminant creates intensities in a band in which oxyhemoglobin reflects more light than deoxyhemoglobin, which is ideal for sensing the former. As can be seen, assuming non-uniform illumination, the red and green can serve as suitable equivalents to the red and near-infrared colors used in traditional pulse oximetry sensors. Therefore, yellow (red+green) is the color displayed by the display. Note that blue may also be used.

To extract the amplitudes of the camera/image color signals, the intensity levels of the red and green channels are obtained from a sensor/image region that is closest to the light source, i.e., the displayed color patch. In one embodiment, ⅓ of the image width for this region is used. In short, a sub-portion of each captured frame may be used as the initial sample. It is also possible to determine a sampling area based on the location where the finger is contacting the display (if the geometry of the smartphone is known in advance). Twenty seconds of camera sampling data may suffice. For each frame or image, a raw value is derived from the sampled region's average intensity, for each color channel. The image area used for processing may also be determined automatically. For example, an image of the finger with and without screen illumination may be compared and only a part of the frame where there is sufficient difference in the signal between the two states/images is used.

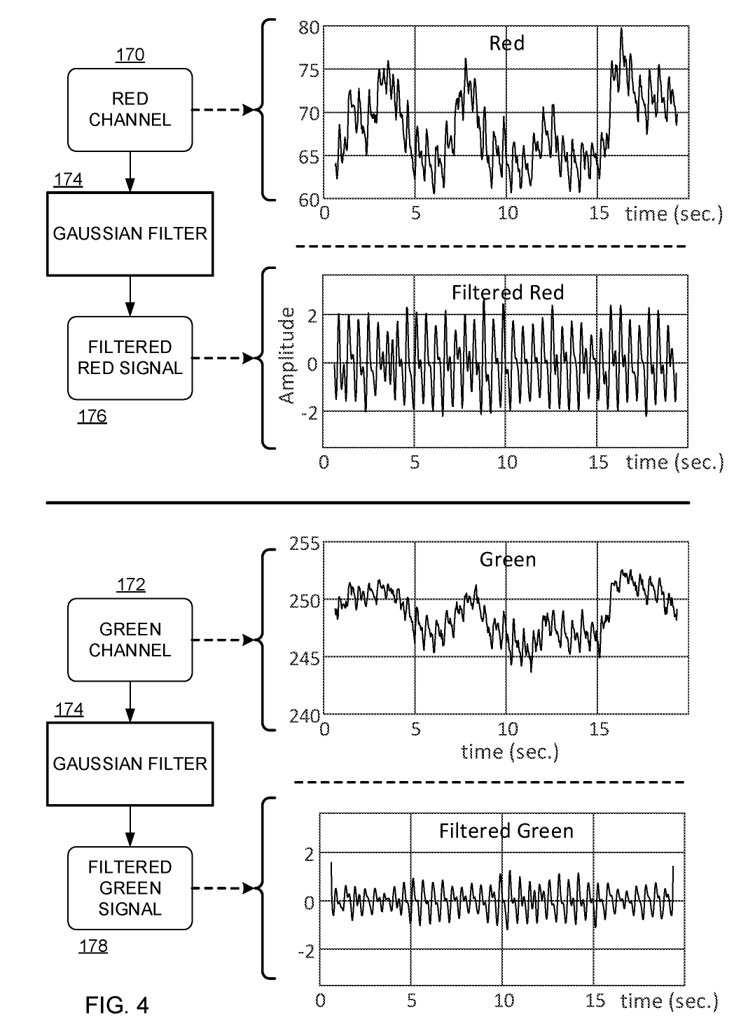

FIG. 4 shows examples of raw and filtered color signals. The red channel 170 is shown at the top of FIG. 4 and the green channel 172 is shown at the bottom of FIG. 4. The raw signals change over time: the red signal band under red display illumination and the green band under green display illumination (albeit not exclusively, as they do overlap, especially towards the near-infrared region). During image acquisition, low frequency changes can be caused by the finger moving slightly, changing touch pressure, or breathing motions. Low frequency changes are removed by applying a first Gaussian filter 174, thus providing filtered red and green signals 176, 178. Note that the camera image may be used to estimate micro-movements of the device relative to the measured body part. For example, when the camera can show skin details, the filtering may also incorporate this motion so that the same signal can be compared despite device-body relative movement. The filtered signals maintain the heartbeat details of the signals. A second low-pass Gaussian filter with a width of ⅕ second, for example, may also be applied to filter noise. From the resulting signals, DC (mean amplitude) and AC (root mean square amplitude) values are computed for the red and green bands, respectively. The AC and DC value may be used to calculate the oximeter ratio R in somewhat known fashion: R=(AC_{Green}/DC_{Green})/(AC_{Red}/DC_{Red}). Formulas using only the AC component are known and may be used instead. Finally, a table mapping R values to blood oxygenation levels is used to obtain the blood oxygenation for the computed ratio R. The R-to-oxygenation lookup table can be created in a clinical study using known techniques.

Although a liquid crystal display was tested, organic light emitting diode displays have similar emission profiles and may provide better contrast. Another approach to illumination is to alternate between displaying red and displaying green. That is, as opposed to displaying red and green together (i.e., yellow), the red channel is obtained only from images captured when the display emits red light and the green channel is obtained only from images captured when the display emits green light. Measurements have demonstrated that using the display as the illuminant provides twice the contrast as using a smartphone flash as the illuminant (assuming similar illumination intensities). Although a smartphone is well-suited to the techniques described herein, any device with suitable processing circuitry and with a display near a camera and both facing the same direction may be used. As noted above, by varying the choice of illuminants, it is possible to determine information about the composition of display-illuminated matter by choosing the illumination colors according to isosbestic points of the illuminated matter; relative changes in the contrast signal can be used to determine relative ratios of constituent components (compounds, elements, etc.) of the target material.

In one embodiment, the color displayed by the display is sized and positioned according to finger position, and low-intensity guides (e.g., lines) are displayed to show where the finger should be placed and kept. Contrast—and hence accuracy and precision—can be improved by minimizing non-display illumination. At the least, covering the device during measurement may be helpful. Performing a measurement in a dark room or measuring with the device may be placed flush against a body area such as forehead or wrist may also increase accuracy. Measurement periods can be communicated to a user using sounds, haptic feedback, or graphics displayed sufficiently distant from the camera.

In yet another embodiment, the captured image/video data is transmitted via a network to another computing device or compute cloud that processes the image/video data to derive a ratio or other measure of constituent components. An application protocol may include elements such as an initial exchange where the device with the camera and display transmit information identifying the device. A backend service and the measuring device both implement the protocol. The backend service maintains a database of devices and their properties, model, and manufacturer, which camera and display each device has, properties of the cameras and displays (e.g. brightness and sensitivity profiles), user instructions for each device, display instructions for displaying color(s), etc. On the measuring device, when a measurement application is registered, installed, or executed, the application sends its identity to the service. The service stores this information in session data, for instance, and returns device-specific information such as display information indicating which color(s) should be displayed, for how long, what patterns or location on the display, etc. When a measurement is taken, the captured image data is sent to the service. The service processes the image data according to the profile of the device and returns the final analysis to the measuring device or smartphone. A final measurement, for instance a percentage of blood oxygenation, is displayed on the display of the measuring device.

[Eyal: In other embodiments, I can imagine someone using a transparent sticker that can accumulate the display light and ‘stream’ it to a point next to the camera, under the finger. Such a contraption might increase the light entering the finger.

In another embodiment I can imagine using a mirror to reflect the display light to the camera. This arrangement could be used to measure transmittance of a liquid between the phone and the mirror.].

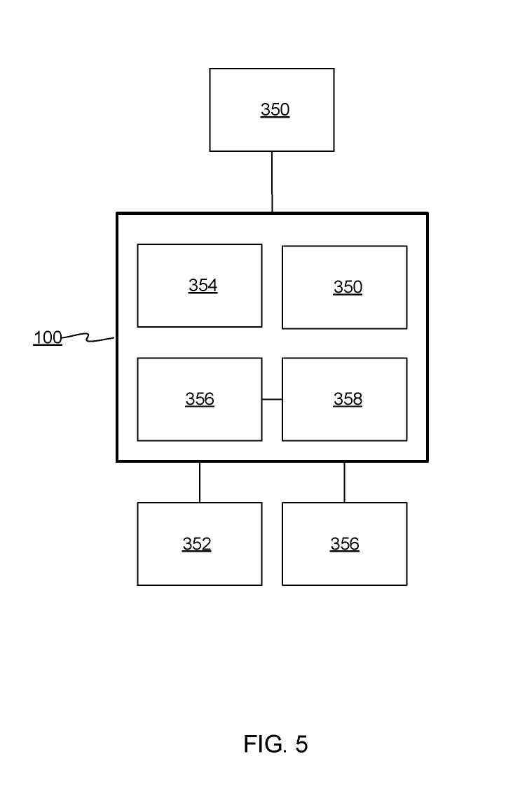

FIG. 5 shows details of the computing device 100 on which embodiments described above may be implemented. The technical disclosures herein will suffice for programmers to write software, and/or configure reconfigurable processing hardware (e.g., field-programmable gate arrays (FPGAs)), and/or design application-specific integrated circuits (ASICs), etc., to run on the computing device 100 to implement any of the features or embodiments described herein.

In addition to the display 104, the computing device 100 may have a network interface 354 (or several), as well as storage hardware 356 and processing hardware 358, which may be a combination of any one or more: central processing units, graphics processing units, analog-to-digital converters, bus chips, FPGAs, ASICs, Application-specific Standard Products (ASSPs), or Complex Programmable Logic Devices (CPLDs), etc. The storage hardware 356 may be any combination of magnetic storage, static memory, volatile memory, non-volatile memory, optically or magnetically readable matter, etc. The meaning of the terms “storage” and “storage hardware”, as used herein does not refer to signals or energy per se, but rather refers to physical apparatuses and states of matter. The hardware elements of the computing device 100 may cooperate in ways well understood in the art of machine computing. In addition, input devices may be integrated with or in communication with the computing device 100. The computing device 100 may have any form-factor or may be used in any type of encompassing device. The computing device 100 may be in the form of a handheld device such as a smartphone, a tablet computer, a gaming device, a server, a rack-mounted or backplaned computer-on-a-board, a system-on-a-chip, or others.

Embodiments and features discussed above can be realized in the form of information stored in volatile or non-volatile computer or device readable storage hardware. This is deemed to include at least hardware such as optical storage (e.g., compact-disk read-only memory (CD-ROM)), magnetic media, flash read-only memory (ROM), or any means of storing digital information in to be readily available for the processing hardware 358. The stored information can be in the form of machine executable instructions (e.g., compiled executable binary code), source code, bytecode, or any other information that can be used to enable or configure computing devices to perform the various embodiments discussed above. This is also considered to include at least volatile memory such as random-access memory (RAM) and/or virtual memory storing information such as central processing unit (CPU) instructions during execution of a program carrying out an embodiment, as well as non-volatile media storing information that allows a program or executable to be loaded and executed. The embodiments and features can be performed on any type of computing device, including portable devices, workstations, servers, mobile wireless devices, and so on.

1. A method of measuring a property of matter, the method performed by a computing device comprising a display, processing hardware, storage hardware, and a camera, the storage hardware storing an operating system that displays graphics on the display, the method comprising:
emitting, from the display, light comprising a first waveband component and a second waveband component, the light transmitting through the matter;
receiving, by the camera, a reflected component of the light transmitted through the matter, the reflected component comprising a reflected first waveband component and a reflected second waveband component, the camera providing a first intensity corresponding to the first reflected waveband component and a second intensity corresponding to the second reflected waveband component; and
computing a value as a function of the first intensity and the second intensity to determine the property of the matter.

2. A method according to claim 1, the matter comprising a first matter component and a second matter component, and wherein the property of the matter comprises a physical ratio of the first matter component to the second matter component.

3. A method according to claim 2, wherein the first matter component and the second matter component have respective different absorption rates for the respective first and second reflected waveband components, wherein the function comprises a ratio of the first intensity and the second intensity, and wherein the ratio varies in proportion to variation in differences between the first and second intensity.

4. A method according to claim 1, wherein the matter comprises flowing blood, and wherein the camera and the display both face the flowing blood.

5. A method according to claim 1, wherein the first and second wavebands of the color are emitted simultaneously by respective different color channels of the display.

6. A method according to claim 1, further comprising computing a pulse rate from the first and/or second intensity.

7. A computing device comprising:
processing hardware;
a camera;
a display; and
storage hardware storing instructions configured to cause the processing hardware to perform a process, the process comprising:
displaying a color by the display;
receiving images from the camera, the images captured by the camera sensing reflected light emitted by the display while displaying the color, the images comprising a first color channel and a second color channel;
computing a first value from the first color channel and computing a second value from the second color channel; and
based on a function value computed as a function of the first value and the second value, determining a measure of a first constituent of a measurement target through which the reflected light transited.

8. A computing device according to claim 7, wherein the measurement target further comprises a second constituent, wherein the first constituent absorbs a first wavelength at a first rate and absorbs a second wavelength at a second rate, wherein the second constituent absorbs the first wavelength at a third rate and absorbs the second wavelength at a fourth rate, wherein the first wavelength corresponds to the first color channel, and wherein the second wavelength corresponds to the second color channel.

9. A computing device according to claim 8, wherein the function comprises a ratio of the first value and the second value, and wherein the measure of the first constituent corresponds to a physical ratio of the first constituent relative to the second constituent.

10. A computing device according to claim 8, wherein the measurement target comprises blood, the first constituent comprises oxyhemoglobin, the second constituent comprises deoxyhemoglobin, and the measure comprises a ratio of oxyhemoglobin to deoxyhemoglobin.

11. A computing device according to claim 7, wherein the displaying the color comprises displaying a patch of color comprising either (i) concurrently, a first color corresponding to the first wavelength and a second color corresponding to the second wavelength, or (ii) alternatively, the first color and the second color.

12. A computing device according to claim 7, wherein the computing device comprises a cell phone and the display comprises a touch-sensitive display.

13. A computing device according to claim 7, further comprising determining, from the images, movements of the computing device relative to the measurement target, and determining the measure based on the determined movements.

14. A computing device according to claim 7, wherein the function value varies in correspondence with contrast between intensities of at least two wavebands of the light that emerges from the measurement target after transiting through the measurement target and reflecting to the camera.

15. A device comprising:
a display facing a direction;
a camera facing in the direction;
processing hardware;
storage hardware storing instructions configured to cause the processing hardware to perform a process comprising:
displaying a first and second color, the first and second colors reflected to the camera;
capturing the colors reflected to the camera by the camera capturing image data while the display is displaying the red and green colors; and
performing a pulse oximetry calculation on the image data to measure blood oxygenation; and
displaying the measure of blood oxygenation on the display.

16. A device according to claim 15, wherein the first color comprises red, the second color comprises green, and wherein a portion of the display displaying the first and second colors does not display blue during the capturing of the colors.

17. A device according to claim 15, wherein the pulse oximetry calculation comprises deriving, from the image data, a first value corresponding to pulsing blood flow in a first color channel and a second value corresponding to pulsing blood flow in a second color channel.

18. A device according to claim 15, wherein a part of a body covers a lens of the camera and also covers a part of the display while the colors are being displayed and while the image data is being captured.

19. A device according claim 15, wherein the process further comprises applying a Gaussian filter to the image data at frequencies substantially outside the range of normal heartrate.

20. A device according to claim 15, wherein the pulse oximetry calculation comprises a ratio comprised of an intensity of the first color and an intensity of the second color.
